(19) United States
(12) Patent Application Publication
(10) Pub. No.: US 2009/0044802 A1
(43) Pub. Date: Feb. 19, 2009

(54) Anesthesia Device

(76) Inventor: Vera Lucia Ferreira Barbosa, Dallas, TX (US)

Correspondence Address: MICHAEL A. O'NEIL, P.C., 5949 SHERRY LANE, SUITE 820, DALLAS, TX, 75225, US

(21) Appl. No.: 12/041,092

(22) Filed: Mar. 3, 2008

Related U.S. Application Data
(63) Continuation-in-part of application No. 11/839,750, filed on Aug. 16, 2007.

(51) Int. Cl. A61M 16/01

(52) U.S. Cl. 128/203.13

(57) ABSTRACT

A device to administer inhalational anesthesia to a user includes a passageway to deliver the anesthesia to the user, a container to hold the anesthesia, a first inlet for inputting the anesthesia into the container, a first unidirectional valve in the passageway to be activated by the user to receive the anesthesia. The device includes a second inlet to deliver the anesthesia to the user, and the second inlet includes a mouthpiece for inhalation and exhalation of anesthetic gases by the user and a second unidirectional valve to control the flow of anesthesia to the atmosphere. The container may be flexible. The passageway may include one or more flexible segment.

### PRIORITY

The present invention is a continuation in part of parent application Ser. No. 11/839,750 which was filed on Aug. 16, 2007.

### BACKGROUND OF THE INVENTION

The present invention relates generally to the field of patient medical masks, such as oxygen masks, and more particularly to an anesthesia device for use by patients in order to eliminate the apprehension associated with patient medical masks that covers or partially covers the face of the patient during the induction of general anesthesia. The induction of anesthesia in children is considered the most stressful period during the entire preoperative period based on behavioral and psychological measures of anxiety (Kain and Mayes, 1996). The induction of general anesthesia in children is commonly done by an inhalation route using anesthetic gases or by an intravenous route. When the inhalation route is used, in some cases, force must be used on the patient to impose the mask on an unwilling patient which may result in a terrifying experience for the patient.

Medical masks, such as oxygen masks, anesthesia masks, respiratory therapy masks, and the like, have long been known in the art. Such masks are typically either made from a somewhat pliable material and/or include a rim of soft rubber-like material. While such masks have proven medically useful, they have also proven to be uncomfortable to some patients and may result in unnecessary apprehension when used for the induction of general anesthesia by inhalation of anesthetic gas. This is particularly true as many potential measures that could be taken to increase patient comfort, decrease fear, anxiety and long-term behavioral and psychological problems may be seen as impeding the critically important operational aspects of the medical device related to its medical function(s).

As such, there remains a need to improve the comfort level of patient medical devices, particularly for the important period of induction of general anesthesia in children.

U.S. Pat. No. 6,698,427 discloses a medical mask assembly that includes a medical mask and a comfort ring releasably mated to the mask. The mask includes an interior surface and an exterior surface and a mating edge where the two meet. The comfort ring includes a strip of cloth and two elastic members secured to the cloth and is formed in the shape of an annular ring with a central aperture therethrough. When mated to the mask, the comfort ring is disposed so as to cover substantially all of the mating edge with one elastic member proximate the interior surface of the medical mask and the other elastic member proximate the exterior surface of the medical mask. As such, the surface touching the patient's face is the outer surface of the comfort ring rather than the interior surface and/or rim edge of the mask.

Anesthesia

U.S. Pat. No. 6,981,503 discloses that during the surgery a patient is usually placed under anesthesia. The simplified common delivery system consists of the canisters containing the anesthesia gases and oxygen, a system of regulating the gas flow and the patient's breathing, and a device ensuring the potency of the patient's airway for breathing, oxygenation and the delivery of the anesthetic gas mixture.

U.S. Pat. No. 5,975,079 discloses a disposable anesthesia mask which has a top portion having an aperture, an encompassed side portion and an open bottom portion. Located on the open bottom portion is an inflatable sealing means, which provides a pneumatic seal between the mask and the patients face. The disposable mask is transparent and is located around the nasal bridge. This shape alleviates orbital pressure. Elongated hooks that are attached to the mask by a head strap plate provides the option of affixing a strap to the mask.

### SUMMARY

The present invention provides for the induction of inhalational general anesthesia by a maskless device (mouthpiece) to reduce the feelings of panic and claustrophobia in patients A device to administer anesthesia to a user includes a passageway to deliver the anesthesia to the user, a container to hold the anesthesia, a first inlet for inputting the anesthesia into the container, a unidirectional valve in the passageway to be activated by the user to receive the anesthesia.

The device includes a second inlet to deliver the anesthesia to the user, and the second inlet includes a valve to control the flow of the anesthesia.

The valve may be controlled by a knob, and the container may be flexible.

The passageway may include a flexible segment.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates the anesthesia device in accordance with the teaching of the present invention;

FIG. 2 illustrates another embodiment of the anesthesia device in accordance with the teachings of the present invention;

FIG. 3 illustrates an open control valve of the present invention;

FIG. 4 illustrates a closed control valve of the present invention;

FIG. 5 illustrates a closed unidirectional valve of the present invention;

FIG. 6 illustrates a open unidirectional valve of the present invention;

FIG. 7 illustrates a closed unidirectional valve of the present invention;

FIGS. 8a and 8b illustrates the operation of the unidirectional valve of the present invention.

### DETAILED DESCRIPTION

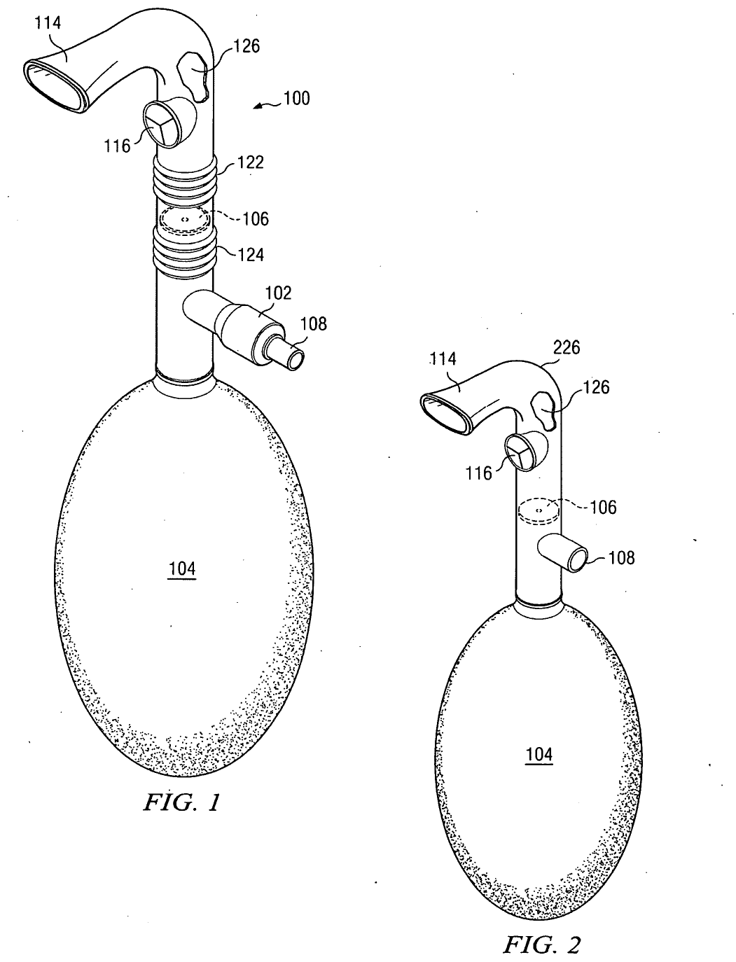

FIG. 1 illustrates an anesthesia device 100 which can be used to the administer anesthesia without the use of a mask. It has been found that using the traditional anesthesia mask being used with patients who do not totally understand the circumstances such as children tends to frighten these patients, resulting in additional problems such as these patients may tend to fight the administering of anesthesia and to have long-term psychological and behavioral problems due to the ‘gorilla induction’ methods commonly used. The anesthesia device 100 of the present invention provides a less intrusive device than the traditional mask and consequently does not tend to frighten patients with each use. The present invention does not use a mask to cover a portion of the patient's face. The present invention includes a passageway 126 which may be a small mouthpiece which is to be used by the patient to inhale the anesthetic gas by his/her own volition (will), and as a result of the small profile of the device, patients are less apprehensive about taking the anesthesia.

FIG. 1 illustrates the anesthesia device 100 which includes a flexible gas (fluid) container 104 and an inlet 102 for anesthesia gas (fluid). The anesthesia fluid is transferred to the flexible fluid container 104 from the inlet 102. The flexible fluid container 104 is a balloon that serves as a reservoir for the anesthesia gas coming through the inlet 102 and the source for the patient to inhale the anesthesia gas from (may be expandable and compressible so that the anesthesia can be first collected to be administered to the patient). The inlet 102 for the anesthesia fluid is shown as being substantially traverse to a passageway 126 which extends from the flexible fluid container 104 to the patient outlet 114 and which is placed into the patient's mouth to receive the anesthesia. The passageway 126 may be a rigid hollow tube and may include a first flexible segment 122 and a second flexible segment 124 so that the passageway 126 can be moved to fit into the patient's mouth. The patient accepts the mouthpiece 114 in his or her mouth in order to ‘blow the balloon’. The mouthpiece 114 may be presented with various tastes palatable to children's taste. Fewer or more flexible segments may be used in connection with the present invention. A unidirectional valve 106 may be positioned within the passageway 126 to prevent the anesthesia gas (fluid) from flowing prematurely to the patient and before the flexible fluid container 104 has substantially been filled with the anesthesia fluid. The unidirectional valve 106 is activated by the patient providing a small amount of suction which opens the unidirectional valve 106 so that the anesthesia fluid can flow from the flexible fluid container 104 through the passageway 126 and to the patient outlet 114 of the passageway 126. The passageway 106 may include a controllable inlet 112 which includes a controllable valve 116 which is controlled by knob 110 to allow exhalation of the anesthesia gas outlet of the patient's lungs. After the patient has been anesthetized by the anesthesia gas the anesthetic device is removed from the anesthesia machine which is to be used in the traditional way.

FIG. 2 illustrates that the passageway 126 includes a curved portion 226 to provide improved access. The curved portion 226 may be at substantially 90° or an angle less than 90°.

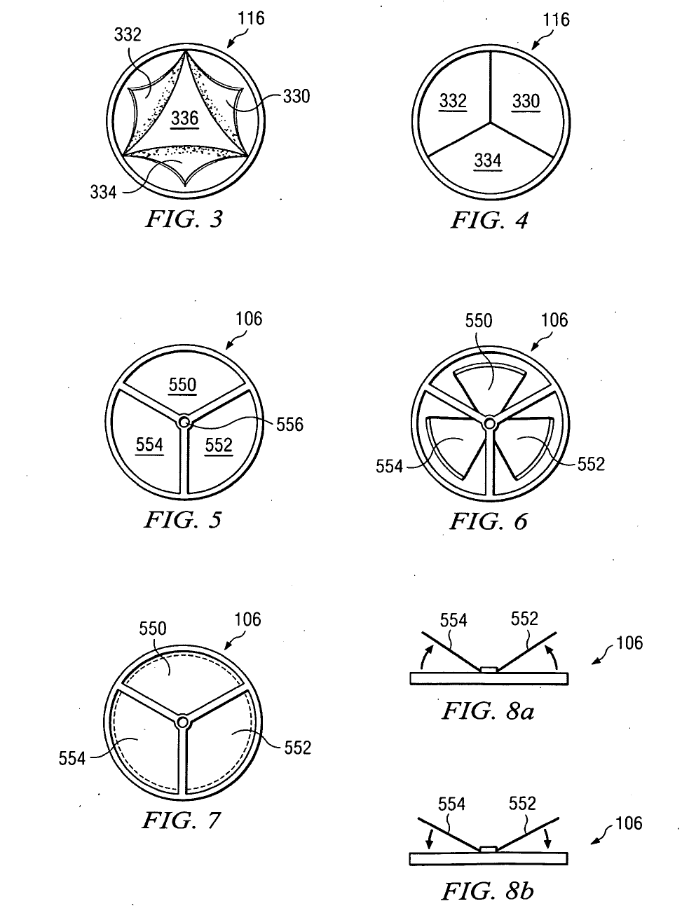

FIG. 3 illustrates an embodiment of the control valve 116 which includes a first leaflet 330, a second leaflet 332 and a third leaflet 334. The edges of the first leaflet 330, the second leaflet 332 and the third leaflet 334 have been opened to form a substantially triangular opening 336 for the control valve 116. The tips of the edges of the leaflets 330, 332, 334 are shown as facing outwards. Other shaped openings 336 are within the scope of the present invention. FIG. 4 illustrates the control valve 116 in a closed position with the first leaflet 332, the second leaflet 334 and the third leaflet 336 covering the opening.

FIG. 5 illustrates the unidirectional valve 106. The unidirectional valve 106 includes a first flap 550 a second flap 552 and a third flap 554. The edges of the first flap 550, the second flap 552 and the third flap 554 forms an approximate Y shape, but other shapes are within the scope of the invention. The unidirectional valve 106 includes a central orifice 556 for the flow of the gases. FIG. 6 illustrates that the first flap 550, the second flap 552 and the third flap 554 are open and up when the gases are flowing out on the balloon generated by inhalation of those gases by the patient. FIG. 7 illustrates that the first flap 550, the second flap 552 and the third flap 554 are closed and down when the flow grasses are out of the patient's mouth by excelling the gases out of lungs. FIG. 8a illustrates a flap 550, 552, 554 opening while FIG. 8b illustrates the flap 550, 552, 55 closing.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1. A device to administer anesthesia to a user, comprising:
a passageway to deliver the anesthesia to the user;
a container to hold the anesthesia;
a first inlet for inputting the anesthesia into the container; and
a first unidirectional valve in the passageway to be activated by the user to receive the anesthesia.

2. A device to administer anesthesia to a user as in claim 1, wherein the device includes a second inlet to deliver the anesthesia to the user.

3. A device to administer anesthesia to a user as in claim 2, wherein the second inlet includes a second unidirectional valve to control the flow of anesthesia and gases during exhalation by the user.

4. (canceled)

5. A device to administer anesthesia to a user as in claim 1, wherein the container is a balloon.

6. A device to administer anesthesia to a user as in claim 1, wherein the passageway includes a flexible segment.

7. A device to administer anesthesia to a user as in claim 1, wherein the passageway includes a curved portion.

8. A device to administer anesthesia to a user as in claim 1, wherein the first unidirectional valve includes a flap.

9. A device to administer anesthesia to a user as in claim 8, wherein the flap is in an upward position when gas vacates the balloon and enters the passageway and into the user's lungs.

10. A device to administer anesthesia to a user as in claim 8, wherein the flap is in a downward position when gas is exhaled from the user.

11. A device to administer anesthesia to a user as in claim 3, wherein the second unidirectional valve includes a plurality of leaflets.

12. A device to administer anesthesia to a user as in claim 11, wherein the plurality of leaflets is in a closed position when gasses flow out of the container.

13. A device to administer anesthesia to a user as in claim 11, wherein the plurality of leaflets is in an opened position when gasses flow out of the user's mouth and into the device.

14. A device for administering a preliminary dose of a predetermined gaseous anesthetic to a juvenile patient comprising:
a reservoir for receiving a gaseous anesthetic;
a substantially imperforate tube of predetermined length extending a predetermined distance from and coupled in fluid communication with the reservoir;
a mouthpiece connected to the tube at the end thereof remote from the reservoir and dimensioned for receipt into the mouth of a juvenile patient;
a first one way valve mounted within the tube for permitting the flow of the gaseous anesthetic from the reservoir through the tube and through the mouthpiece into the mouth of the juvenile patient; and
a second one way valve mounted on the tube at a location between the first one way valve and the mouthpiece for permitting gases exhaled by the juvenile patient to flow through the mouthpiece and through the portion of the tube extending between the mouthpiece and the second one way valve and through the second one way valve into the atmosphere while preventing gases comprising the atmosphere from entering the tube.

15. The device for administering a preliminary dose of a predetermined gaseous anesthetic to a juvenile patient of claim 14 wherein the first one way valve comprises a plurality of flaps, thereby permitting the flow of gaseous anesthetic from the reservoir to the mouthpiece and into the juvenile patient's lungs upon suction by the juvenile patient.

16. The device for administering a preliminary dose of a predetermined gaseous anesthetic to a juvenile patient of claim 14 wherein the second one way valve comprises a plurality of leaflets, thereby permitting gases exhaled by the juvenile patient to flow through the mouthpiece and through the portion of the tube extending between the mouthpiece and the second one way valve and through the second one way valve into the atmosphere while also preventing gases comprises the atmosphere from entering the tube.

17. The device for administering a preliminary dose of a predetermined gaseous anesthetic to a juvenile patient of claim 14 wherein the tube comprises a curved portion.

18. The device for administering a preliminary dose of a predetermined gaseous anesthetic to a juvenile patient of claim 14 wherein the reservoir is a balloon.

19. The device for administering a preliminary dose of a predetermined gaseous anesthetic to a juvenile patient of claim 14 wherein the tube comprises a flexible segment.

20. The device for administering a preliminary dose of a predetermined gaseous anesthetic to a juvenile patient further comprising an inlet for inputting the gaseous anesthetic into the reservoir while preventing the gaseous anesthetic therein from escaping therethrough to the atmosphere.
